(19) United States
(12) Patent Application Publication
(10) Pub. No.: US 2021/0137919 A1
(43) Pub. Date: May 13, 2021

(54) PHARMACEUTICAL FORMULATIONS, METHOD FOR PRODUCING A PHARMACEUTICAL FORMULATION, AND MEDICAMENT COMPRISING SAME

(71) Applicant: SmaWa GmbH, Wurzburg (DE)

(72) Inventors: Norbert Roewer, Wurzburg (DE); Jens Broscheit, Wurzburg (DE); Isabell Steer, Essen (DE)

(21) Appl. No.: 16/968,091

(22) PCT Filed: Feb. 7, 2019
(86) PCT No.: PCT/EP2019/052972

§ 371 Date: Jan. 22, 2021

(30) Foreign Application Priority Data
Feb. 7, 2018 (EP) 18155542.6

(51) Int. Cl. A61K 31/4985; A61K 47/32; A61K 47/38; A61K 47/10; A61K 47/20; A61K 9/00; A61K 9/10; A61P 15/10

(52) U.S. Cl. CPC A61K 31/4985; A61K 47/32; A61K 47/38; A61K 47/10; A61P 15/10; A61K 9/006; A61K 9/10; A61K 9/0043; A61K 47/20

(57) ABSTRACT

The invention provides a pharmaceutical buccal, sublingual, gingival or intranasal formulation comprising tadalafil or salt thereof as active substance, a polymer, and a surfactant, and also a method for the production thereof and the use of the formulation in a medicament for treating sexual dysfunction. The average particle size of the active substance is within a range from 8 to 500 nm and the polymer is polyvinylpyrrolidone (PVP) and/or vinylpyrrolidone-vinyl acetate copolymer (KVA). The surfactant may, for example, be sodium dodecyl sulfate (SDS). The maximum serum active substance concentration occurs within just 1 hour after administration of the medicament.

The invention relates to a pharmaceutical formulation, to a method for producing a pharmaceutical formulation, to a pharmaceutical formulation obtainable by such a method, and to a medicament comprising a pharmaceutical formulation.

The active substance tadalafil (IUPAC name: ((6R,12aR)-6-(1,3-benzodioxol-5-yl)-2-methyl-1,2,3,4,6,7,12,12a-octahydropyrazino-[2′,1′:6,1]pyrido[3,4-b]indol-1,4-dione) belongs to the group of PDE-V (phosphorodiesterase V) inhibitors, which are used as an oral formulation for the treatment of erectile dysfunction. The medicament is given in the form of a tablet and is absorbed intestinally, that is to say via the intestine. Absorption via the intestine takes place through the intestinal mucosa of the small intestine (jejunum). On the one hand, tadalafil belongs to the group of “small molecules” (molecular weight=389 g/mol) having lipophilic properties (log P=+1.7) and as such is able to diffuse freely through any membrane. On the other hand, tadalafil needs to be able to reach the membrane and accordingly be soluble in the aqueous medium found there. Because of its strong tendency to crystallize, tadalafil has a solubility in water of only about 2-3.2 μg/ml and therefore belongs to the group of substances that are poorly soluble in water.

The bioavailability of orally administered tadalafil is directly dependent on the degree of solubility in water. In order for oral formulations of tadalafil to have sufficiently high bioavailability despite the poor solubility of the active substance, it is necessary to improve the solubility in water. An important step in this direction is breaking up the crystalline structure of the solid by micronization, that is to say significant mechanical reduction of the average particle size to below 200 μm. A classic method for producing such fine particles is milling. The success of this measure does, however, depend on the extent to which it is possible to prevent recrystallization of the micronized substance in aqueous solution.

What is problematic here is that the micronized particles have a tendency to form agglomerates. This results in a nonuniform distribution of particle sizes, which is in turn reflected in varying solubilities. An electrostatic charge on the active substance resulting from the comminution process also has an adverse effect on processability. A further potential disadvantage is the poor flowability of the milled active substance. In particular, when the active substance is to be pressed into tablets or filled into capsules, this means that further processing steps such as granulation are required. Pharmacokinetic data in US patent application 2015/0359735 A1 disclose that the maximum serum concentration (tₘₐₓ) of tadalafil active substance when using a film produced from a suspension comprising micronized tadalafil, surfactant, and cellulose-based polymers is reached no earlier than 2.50 hours after administration. This long time is unsatisfactory, particularly when treating sexual dysfunction with the active substance tadalafil, for example in the context of treatment of acute erectile dysfunction, in which a rapid onset of the therapeutic outcome and/or prophylactic use to ensure adequate erectile function while prioritizing the shortest possible latency time is desirable. “Latency time” here is the period of time from ingestion of a medicament to the onset of action.

The object underlying the invention is accordingly to create a pharmaceutical formulation in which the solubility and rate of dissolution of tadalafil or salt thereof as active substance are improved, thereby resulting in increased pharmacological efficiency through a more rapid onset of action and increased bioavailability.

This object is achieved by a pharmaceutical formulation as claimed in the main claim. The invention thus relates in particular to a pharmaceutical buccal, sublingual, gingival or intranasal formulation comprising the following components:
a) tadalafil or salt thereof as active substance, the average particle size of the active substance in said formulation being within a range from 8 to 500 nm,
b) a polymer, said polymer being polyvinylpyrrolidone (PVP) and/or vinylpyrrolidone-vinyl acetate copolymer (KVA), and
c) a surfactant.

Further advantageous embodiments can be found in the subclaims.

To begin with, some terms used in the context of the invention will be explained.

In the context of the invention described in more detail hereinbelow, the term “micronizing polymer”, which also encompasses the abovementioned polymers PVP and KVA, is understood as meaning a polymer that is used together with the surfactant for the comminution of the active substance particles. A distinction must be made here from the “film-forming polymer” described in the context of the invention, which is used for the production of films. It should be noted that a few polymers, such as hydroxypropyl methylcellulose in particular, are able to perform a dual role, allowing them to be used in the context of the invention as both micronizing polymer and film-forming polymer. However, this is not normally the case.

The invention has recognized that the addition of a combination of micronizing polymer, in particular PVP and/or KVA, and surfactant allows problem-free stabilization of comminuted active substance particles having an average particle size of well below 500 nm, in particular below 390 nm. Surprisingly, the problems described in the prior art, such as recrystallization and the formation of agglomerates and/or the build-up of electrostatic charge on the active substance, do not occur in the pharmaceutical formulation according to the invention. This is because whereas the micronizing polymer contributes considerably to a steric stabilization of the active substance, the addition of the surfactant (SDS) counteracts the undesired build-up of electrostatic charge on the active substance particles. The pharmaceutical formulation of the invention can therefore be handled in a straightforward manner and accordingly shows improved storage stability, considerably increased general solubility, and a higher rate of dissolution of the active substance. The increased solubility of the active substance in turn leads, particularly when using PVP and KVA, to a surprising improvement in pharmacological efficiency due to a more rapid onset of action and increased bioavailability. It should at this point be noted that the specific combination of the claimed components a), b), and c) achieved tₘₐₓ, i.e. reached the peak plasma tadalafil concentration (cₘₐₓ), after less than 40 minutes, and thus many times more rapidly than with the conventional administration of a commercially available Cialis® tablet (see FIG. 4) or of the film disclosed in US 2015/0359735, which contains cellulose-based polymers in addition to the active substance tadalafil and surfactant, but not PVP or KVA.

In the context of the invention, it is preferable that the average particle size of the active substance is within a range from 10 to 390 nm, more preferably within a range from 100 to 390 nm, most preferably within a range from 200 to 350 nm.

It is further preferable that, in addition to the micronizing polymers PVP and/or KVA, further polymers may be included, selected from hydroxypropyl methylcellulose, hydroxypropyl cellulose, methylcellulose and mixtures thereof.

It is further preferable that the surfactant is an anionic surfactant. Even more preferably, the anionic surfactant is selected from alkyl sulfates, alkyl sulfonates, aryl sulfates, aryl sulfonates, and mixtures thereof. In a particularly preferred embodiment, the anionic surfactant is sodium dodecyl sulfate.

Particularly preferred pharmaceutical formulations are those in which, in addition to the tadalafil active substance component a), the polymer component and surfactant component are selected as follows:
said polymer according to component b) PVP and said surfactant according to component c) SDS, or
said polymer according to component b) KVA and said surfactant according to component c) SDS, or
said polymer according to component b) a mixture of PVP and KVA and said surfactant according to component c) SDS.

In addition, it is preferable that the pharmaceutical formulation is selected from the group consisting of
i) a film,
ii) an aerosol,
iii) an aqueous suspension, solution, tincture, cream, paste, lotion, ointment, gel, or capsule releasing these formulations in the oral cavity,
iv) an orodispersible tablet, lozenge or buccal tablet, the abovementioned formulations preferably being mucoadhesive formulations (formulations that adhere to mucous membranes).

In these pharmaceutical formulations, the tadalafil active substance in the formulation is supplied to the bloodstream for a systemic action via the mucosa of the oral cavity or nose. An important advantage in particular is that the active substance is able to circumvent the gastrointestinal tract, thereby avoiding the longer latency time associated with conventional Cialis® tablets, and can also be given without intravenous administration, which is both uncomfortable and can be accompanied by a host of undesirable problems (dosage, infections, etc.) if executed incorrectly.

In a preferred embodiment, a maximum serum active substance concentration is reached in vivo within not more than 120 minutes, more preferably within not more than 90 minutes, even more preferably within not more than 60 minutes, after administration of the pharmaceutical formulation.

It is further preferable that further constituents of the pharmaceutical formulation are selected from plasticizers and film-forming polymers. An example of a preferred plasticizer is glycerol. An example of a preferred film-forming polymer is hydroxypropyl methylcellulose.

It is also preferable that a content of surfactant in the pharmaceutical formulation is 0.001% to 0.5% by weight, more preferably 0.01% to 0.3% by weight, even more preferably 0.025% to 0.1% by weight, based on the overall composition of the pharmaceutical formulation.

It is further preferable that a content of micronizing polymer in the pharmaceutical formulation is 0.1% to 2% by weight, more preferably 0.5% to 1% by weight, based on the overall composition of the pharmaceutical formulation.

It is preferable that a content of active substance in the pharmaceutical formulation is 0.5% to 5% by weight, more preferably 2% to 3% by weight, based on the overall composition of the pharmaceutical formulation.

It is further preferable that a content of film-forming polymer in the pharmaceutical formulation is 2% to 30% by weight, more preferably 7% to 17% by weight, based on the overall composition of the pharmaceutical formulation.

It is also preferable that a content of plasticizer in the pharmaceutical formulation is 1% to 20% by weight, more preferably 2% to 8% by weight, based on the overall composition of the pharmaceutical formulation.

It is preferable that a content of water in the pharmaceutical formulation is 20% to 95% by weight, more preferably 40% to 85% by weight, based on the overall composition of the pharmaceutical formulation.

The invention further provides a method for producing a pharmaceutical formulation as described above, wherein the active substance tadalafil is comminuted at least together with the micronizing polymer (PVP and/or KVA) and a surfactant (for example SDS). In the context of the invention, comminution of the active substance at least together with the micronizing polymer and the surfactant means that the micronizing polymer and the surfactant must already be present in the composition alongside the active substance during the comminution step of the method. Further constituents of the pharmaceutical formulation may already be present in the pharmaceutical formulation during comminution of the active substance or may be added at a later time after comminution.

As already described above, it is preferable that, in addition to the micronizing polymers polyvinylpyrrolidone and/or vinylpyrrolidone-vinyl acetate copolymer, the formulation also contains hydroxypropyl methylcellulose, hydroxypropyl cellulose, methylcellulose or mixtures thereof.

It is further preferable that the surfactant is an anionic surfactant. Even more preferably, the anionic surfactant is selected from alkyl sulfates, alkyl sulfonates, aryl sulfates, aryl sulfonates, and mixtures thereof. In a particularly preferred embodiment, the anionic surfactant is sodium dodecyl sulfate.

It is further preferable that the average particle size of the active substance after comminution is within a range from 8 to 500 nm, more preferably within a range from 10 to 390 nm, even more preferably within a range from 100 to 390 nm, most preferably within a range from 200 to 350 nm.

The comminution is preferably carried out for a period of 100 to 260 minutes, preferably for a period of 140 to 180 minutes.

In addition, it is preferable that comminution is a milling process, more preferably wet milling.

It is further preferable that comminution takes place in a ball mill, more preferably in a stirring ball mill, with milling in the stirring ball mill preferably carried out at a peripheral stirrer speed of more than 4 m/s, preferably 5-15 m/s, more preferably 7-11 m/s, particularly preferably 9 m/s.

Further components may be added to components a), b), and c) of the pharmaceutical formulation during and/or after the combined comminution thereof. For example, it is possible that
i) after combined comminution of components a), b), and c) in a stirring ball mill, further components to produce a film are added to the stirring ball mill, said further components preferably comprising water-soluble cellulose derivatives,
ii) the resulting total mixture in the stirring ball mill is homogenized, and then
iii) the homogenate obtained is applied to a film as a coating compound or is itself processed into a film.

The abovementioned homogenization step ii) may be carried out in the stirring ball mill at a peripheral stirrer speed of more than 2 m/s, preferably 3-12 m/s, more preferably 4-8 m/s, particularly preferably 6 m/s.

It is further preferable that a content of surfactant in the pharmaceutical formulation is 0.001% to 0.5% by weight, more preferably 0.01% to 0.3% by weight, even more preferably 0.025% to 0.1% by weight, based on the overall composition of the pharmaceutical formulation.

The invention further provides a pharmaceutical formulation obtainable by the method according to the invention for producing a pharmaceutical formulation as described above.

The invention further provides a medicament comprising the pharmaceutical formulation according to the invention or the pharmaceutical formulation obtainable by the method according to the invention. The medicament may be formulated as previously described for the pharmaceutical formulation, for example as an orodispersible tablet, lozenge or buccal tablet, a capsule that releases an aqueous suspension, solution, tincture, cream, paste, lotion, ointment or gel contained therein into the oral cavity, or as such formulations on their own in unencapsulated form, an aerosol or as a film. Particular preference is given to the formulation in the form of a film. This is because the abovementioned properties of the pharmaceutical formulation according to the invention allow it to be distributed homogeneously on a film. Moreover, oral administration or oral intake of a film allows particularly rapid absorption of active substances through the oral mucosa.

The invention further provides the medicament according to the invention for use in the treatment of sexual dysfunction, preferably erectile dysfunction.

In addition, it is preferable that a maximum serum active substance concentration (tₘₐₓ) is reached in vivo within not more than 120 minutes, more preferably within not more than 90 minutes, even more preferably within not more than 60 minutes, particularly preferably within not more than 45 minutes, after administration of the pharmaceutical formulation.

The invention will now be explained on the basis of advantageous embodiments with reference to the included drawings. In the figures:

FIG. 1: shows the test results for the comminution of tadalafil with additives.

FIG. 2: shows the change in particle size during comminution of tadalafil.

FIG. 3: shows release profiles of tadalafil film preparations versus the commercial product Cialis® (5 mg).

FIGS. 4-7: show plots (y-axis=concentration (ng/ml); x-axis=time (min)) of the test results of the crossover study for the determination of the plasma concentrations.

### 1. INVESTIGATION OF THE COMMINUTION OF TADALAFIL WITH ADDITIVES

The investigations of the formulation of tadalafil against particle agglomeration during comminution were carried out in a planetary ball mill (PM400, Retsch). A total of 11 different steric and electrosteric formulations were tested. This was done using milling compartments made of zirconium oxide and having a milling compartment volume of 1 ml. The differently formulated suspensions were comminuted using yttrium-stabilized zirconium oxide (ZrO₂, Sigmund Lindner) milling media having a diameter of d_{50,MM}=475 μm, the filling level of the milling media being φ_{MM}=0.5. The tadalafil particles were stressed in the mill for 2 hours at a sunwheel speed of vₛᵤₙ=400 rpm. The suspensions were then measured by dynamic light scattering (Nanophox, SympaTec). The principle of dynamic light scattering (DLS) is employed for the characterization of particle sizes and is based on the detection of the scattered light intensity of particles in thermal motion. For this, 200 μl of the respective suspension was diluted in 1 ml of distilled water. The diluted suspension was then transferred to an acrylic glass cell and placed in the beam path of the instrument for measurement at room temperature and standard pressure.

The additives used for the formulations, the abbreviations thereof, and suppliers of the additives are shown in Table 1.

TABLE 1
Tested additives

| Additive | Abbreviation | Supplier |
|---|---|---|
| Polyvinylpyrrolidone | PVP | Sigma |
| | | Aldrich |
| Vinylpyrrolidone-vinyl acetate | KVA | BASF |
| copolymer (Kollidon ®VA64) | | |
| Hydroxypropyl cellulose | HPC | Sigma |
| | | Aldrich |
| Hydroxypropyl methylcellulose | HPMC | ShinEtsu |
| Methylcellulose | MC | Sigma |
| | | Aldrich |
| Sodium dodecyl sulfate | SDS | Sigma |
| | | Aldrich |

Formulations were prepared from the abovementioned additives in distilled water. The tadalafil concentration in the suspension was always cₘ=0.05. For the sterically stabilized formulations, an additive concentration of c_{add}=0.4 was chosen (all additive contents are based on the solids concentration). In the electrosterically stabilized samples, the polymer content was c_{poly}=0.3 and the surfactant content was c_{surfactant}=0.1. The test results are shown in FIG. 1. All results are based on a duplicate determination.

Comminution without additive was carried out as a reference, which shows clearly that an average particle size of below x₅₀=7 μm cannot be achieved without particle stabilization. The comminution results show that a particle size of below x₅₀=700 nm can be achieved in a comminution time of 2 hours with almost all selected additives. With the celluloses HPC and MC, average particle sizes of below x₅₀=410 nm can be achieved. However, the best results are achieved only through the additional addition of surfactant, which counteracts the build-up of electrostatic charge and thus results in further stabilization of the tadalafil particles. The additional charge on the particle brought about by the surfactant provides an even more efficient stabilization against agglomeration during comminution. The most suitable formulation, which achieved an average particle size of x₅₀=230 nm, is the combination of the polymer PVP and the surfactant SDS.

### 2. COMMINUTION OF TADALAFIL

The pharmaceutical active substance tadalafil (x₅₀=6.4 μm) was comminuted in a stirring ball mill (MiniCer, Netzsch) with two different formulations comprising the polymer PVP or KVA and the surfactant SDS. The trade names of the additives and the suppliers are given in Table 1.

In order to achieve an active substance loading of 10 mg in a 6 cm² orodispersible film (ODF), a solids content (mₜₒₜₐₗ=500 g) of cₘ=0.033 was initially charged in the suspension. A polymer concentration c_{poly}=0.25 and a surfactant concentration c_{SDS}=0.025 (both based on the solids content in the suspension) were added to stabilize the suspension against agglomeration during comminution. The process parameters used during comminution are shown in Table 2.

TABLE 2
Process parameters during comminution in the stirring ball mill

| | | |
|---|---|---|
| General parameters | Milling media material | Zirconium |
| | | oxide |
| | Milling media size | d_{MM} = 325 μm |
| | Filling level of milling media | φ_{MM} = 0.8 |
| Comminution | Peripheral stirrer speed | vₜ = 9 m/s |
| | Comminution time | t_{comm} = |
| | | 140 min or |
| | | 180 min |
| Homogenization | Peripheral stirrer speed | vₜ = 6 m/s |
| | Homogenization time | tₕₒₘₒ = 20 min |

FIG. 2 shows the change in the average particle size during the comminution process in relation to the specific energy input of the mill. For both formulations, a rapid increase in particle fineness is observed within the first minutes of comminution, from an initial particle size x₅₀=6.4 μm to below x₅₀=430 nm. With increasing comminution time, the average particle size falls, up to an energy input of Eₘ=55 000 kJ kg⁻¹, to 267 nm for the formulation with KVA and SDS and to 342 nm for the formulation with PVP and SDS. Slight particle agglomeration is subsequently observed, which is attributable to the stabilization of the particles as a consequence of the newly formed surfaces no longer being sufficient. It is clear that the formulation with KVA and SDS is particularly suitable for the tadalafil particles in aqueous suspension, since smaller average particle sizes can be achieved with the same energy input during comminution.

After completion of the comminution, 115 ml of the suspension was removed from the mill for further tests. The remaining suspension was processed directly into a coating compound in the mill. This was done using the method of Steiner et al., “Efficient production of nanoparticle-loaded orodispersible films by process integration in a stirred media mill”, International Journal of Pharmaceutics, 2016, vol. 511, pp. 804-813. The film-binding polymer HPMC (Pharmacoat 606, ShinEtsu) was added directly to the suspension in the mill (c_{HPMC}=0.15) and homogenization was carried out for 20 min once the polymer had dissolved. The plasticizer glycerol (c_{gly}=0.05) was then added and the coating composition homogenized again for a further 5 minutes in the mill. The sample was then removed from the mill and sealed airtight.

### 3. PRODUCTION OF FILMS

### Materials

TABLE 3
List of materials used

| Substance | Abbreviation | Supplier |
|---|---|---|
| Tadalafil | | |
| Polyvinylpyrrolidone | PVP | Sigma |
| | | Aldrich |
| Vinylpyrrolidone-vinyl acetate | KVA | BASF |
| copolymer (Kollidon ®VA64) | | |
| Hydroxypropyl methylcellulose | HPMC | ShinEtsu |
| (Pharmacoat 606 ®) | | |
| Sodium dodecyl sulfate | SDS | Sigma |
| | | Aldrich |
| Glycerol | Gly | Caelo |
| Distilled water | | |

### Film Production

Dose per film: 8.12 mg
Film thickness (wet): 500 μm
Size of a film: 6 cm²

TABLE 4
Composition of the film preparations

| | Tadalafil, | Tadalafil, |
|---|---|---|
| Substance | KVA & SDS | PVP & SDS |
| Tadalafil | 2.73% | 2.73% |
| PVP | — | 0.679% |
| KVP | 0.679% | — |
| SDS | 0.068% | 0.068% |
| HPMC | 12.43% | 12.43% |
| Glycerol | 4.75% | 4.75% |
| Water | 79.33% | 79.33% |

a) Comminution of the Active Substance

The active substance was comminuted in a stirring ball mill (MiniCer, Netzsch, Germany). For this, the polymer PVP or KVA for steric stabilization corresponding to the respective formulation and SDS for electrostatic stabilization were dissolved in distilled water with stirring. The active substance tadalafil was then added. The suspension comprising tadalafil, KVA and SDS was wet-milled for 140 min and the suspension comprising tadalafil, PVP and SDS was wet-milled for 180 min using zirconium oxide (325 μm; 80% filling with milling media) at a speed of 9 m/s.

b) Production of the Films

After the respective comminution of the active substance, the film-forming polymer HPMC and the plasticizer glycerol were added to the respective suspensions in the stirring ball mill. The mixtures were homogenized for a further 20 min at a speed of 6 m/s. The mixtures were stirred slowly (50 rpm) for 12 hours to remove air bubbles. The films were produced at a blade height of 500 μm using an automated film-drawing bench (Coatmaster 500, Erichsen) on a polyethylene terephthalate film at a speed of 6 mm/s at room temperature. After drying the films for 12 hours at room temperature, the films produced were manually cut into rectangular pieces (2×3 cm).

### Methods of Analysis

a) Particle Size Determination

The particle size distribution of the suspensions was determined by dynamic light scattering (Nanophox, SympaTec). For this, approx. 200 μl of the respective film suspension was diluted in 2 ml of distilled water and measured at room temperature and standard pressure.

b) Dissolution

The dissolution tests were carried out in 900 ml of distilled water at 37±0.5° C. using the paddle stirrer apparatus (DT700, Erweka, Ph. Eur. Apparatus 1) at a speed of 100 rpm. For this, the films were affixed with double-sided adhesive tape to a glass plate (ø7 cm), which was laid on a vessel base such that the films were positioned centrally beneath the paddle stirrer. In the case of the known Cialis® tablets (5 mg), affixing to the glass plate was omitted. Sampling was carried out manually at the specified times. The samples (4 ml) were collected with a 5 ml disposable syringe (Soft-Ject® 5 ml) through a frit (pore diameter 100 μm) and through a syringe filter (Puradisc® 25, Whatman, PVDF membrane, ø25 mm, pore diameter 0.2 μm), filtered into an HPLC vial, discarding the first 3 ml of the filtrate. The liquid removed from the vessel was replaced with 4 ml of preheated (37±0.5° C.) distilled water. The filter was replaced after collection of every 5 samples. The sample concentration was determined by HPLC-MS/MS.

### Results

a) Particle Size Determination

The initial particle size was 6.4 μm (x50% (Q3)). The X50 (Q3) denotes the average particle size (median particle size) determined from a cumulative distribution Q3 based on determination of the particle volume using the following formula:

$$Cumulative frequency Q 3 i = Σ \frac{Δ {M}_{i}}{{M}_{total}} * 100$$

The average particle size was reduced during comminution to below 430 μm (x50% (Q3)) after just a few minutes. The particle size was reduced to 267 nm (x50% (Q3)) in the case of tadalafil in combination with KVA and SDS and to 342 nm (x50% (Q3)) for tadalafil in combination with PVP and SDS (see FIG. 2).

b) Dissolution

The dissolution tests were in all cases carried out under non-sink conditions, i.e. the saturation concentration of tadalafil in the aqueous medium was exceeded, with the result that a saturated solution with a precipitate formed. The amount of tadalafil released accordingly depends on the solubility of the active substance in the dissolution medium (distilled water). FIG. 3 shows the dissolution curves of the two film preparations versus that of the known Cialis® tablets (5 mg). This shows that the dissolution rates of the different formulations barely differ, since the film formulations and the tablet are both pharmaceutical forms that disintegrate rapidly. The film preparations release the active substance tadalafil somewhat more rapidly (within the first 60 minutes) than the commercial product Cialis®. However, the film preparations can be seen clearly to reach considerably higher maximum concentrations. This is particularly pronounced in the films comprising tadalafil, KVA, and SDS. This can be attributed to the considerable reduction in size of the active substance particles in the films, which increases general solubility considerably, resulting in a higher rate of dissolution of the active substance.

### Permeation Studies in Healthy Volunteers

a) Methodology

Inclusion criteria for the study were: age over 18 years and absence of serious comorbidities. The exclusion criterion for the study was detection of tadalafil at the time of commencement of the study phase (t0).

b) Study Medication Pharmacokinetics

The following products were used in the studies:
As a comparison, the commercial product Cialis® from Lilly Pharma, 10 mg (usual dose for the treatment of erectile dysfunction)
Film comprising tadalafil, KVA, and SDS, production as described above under film production, 8 mg
Film comprising tadalafil, PVP, and SDS, production as described above under film production, 8 mg

c) Crossover Study—Determination of Plasma Concentrations

A crossover study allows comparison of target parameters such as treatment forms or plasma concentrations. In such studies, the investigated agents are administered sequentially to the same subjects. Compared to conventional studies with parallel comparison groups, crossover studies have the advantage that smaller differences in the target parameters (e.g. plasma concentrations) become statistically significant or that fewer participants are required to demonstrate a significant difference.

In a crossover study, account must be taken of the so-called carryover effect, i.e. carryover of the effects of administration of the first agent into the following administration phase. To ensure an effect of the first agent is no longer present, there therefore needs to be an interval between the treatment phases during which nothing is administered. The plasma active substance concentration is therefore determined at the start of each study phase (time to) and the study findings are evaluated if no active substance is detectable, that is to say t₀=0.

### Results:

FIGS. 4 to 7 show plots (y-axis=concentration (ng/ml); x-axis=time (min)) of the test results of the crossover study for the determination of the plasma concentrations.

FIGS. 4 and 5 show the average values for the serum concentrations in 4 different subjects. Line A represents the serum concentration after a dose of 8 mg of tadalafil formulated in the film-coated tablet (ODF) with the micronizing polymer PVP and the surfactant SDS, whereas in FIG. 4 this line is independently labeled, with inclusion of the tₘₐₓ value of <40 min for a swifter overview. In FIG. 4, the serum concentration after a dose of 8 mg of tadalafil formulated in the film-coated tablet (ODF) with the micronizing polymer KVA and the surfactant SDS is similarly labeled, likewise with inclusion of the tₘₐₓ value of <40 min for a swifter overview. Line B in FIG. 5 represents the serum concentration after a dose of 10 mg of tadalafil formulated in a commercially available tablet of Cialis® (Lilly Pharma), whereas in FIG. 4 this line is independently labeled, with inclusion of the tₘₐₓ value of >240 min for a swifter overview.

FIGS. 6 and 7 show individual values for the individual subjects DS and JB. Lines A represent the serum concentration after a dose of 8 mg of tadalafil formulated in the film-coated tablet (ODF) with the micronizing polymer PVP and the surfactant SDS. Lines B represent the serum concentration after a dose of 10 mg of tadalafil formulated in a commercially available tablet of Cialis® (Lilly Pharma).

### CONCLUSION

In the exemplary embodiments, it was shown that the addition of a combination of micronizing polymer and surfactant allows the problem-free stabilization of comminuted active substance particles having an average particle size of well below 500 nm, in particular below 390 nm, when using a stirring ball mill. The inline production of the film mass in the stirring ball mill according to the method of Steiner et al., “Efficient production of nanoparticle-loaded orodispersible films by process integration in a stirred media mill,” International Journal of Pharmaceutics, 2016, vol. 511, pp. 804-813, is easily implemented. The films can be produced without problem in this way. Corresponding tests of the dissolution of the active substance show that both the general solubility of the active substance tadalafil in aqueous media and the rate of dissolution can be increased considerably by comminution of the active substance particles. In further studies, it was additionally shown that this results in an increase in pharmacological efficiency through a more rapid onset of action and increased bioavailability when PVP or KVA are used.

1. A pharmaceutical buccal, sublingual, gingival or intranasal formulation comprising the following components:
a) tadalafil or salt thereof as active substance, the average particle size of the active substance in said formulation being within a range from 8 to 500 nm,
b) a polymer, said polymer being polyvinylpyrrolidone (PVP) and/or vinylpyrrolidone-vinyl acetate copolymer (KVA), and
c) a surfactant.

2. The pharmaceutical formulation as claimed in claim 1, characterized in that the average particle size of the active substance according to component a) is within a range from 10 to 390 nm, more preferably within a range from 100 to 390 nm, most preferably within a range from 200 to 350 nm.

3. The pharmaceutical formulation as claimed in claim 1, characterized in that said formulation comprises, in addition to the polymer according to component b), at least one further polymer selected from hydroxypropyl methylcellulose, hydroxypropyl cellulose, methylcellulose, and mixtures thereof.

4. The pharmaceutical formulation as claimed in claim 1, characterized in that the surfactant according to component c) is an anionic surfactant, preferably an anionic surfactant selected from alkyl sulfates, alkyl sulfonates, aryl sulfates, aryl sulfonates, and mixtures thereof, more preferably sodium dodecyl sulfate (SDS).

5. The pharmaceutical formulation as claimed in claim 1, characterized in that
said polymer according to component b) are PVP and said surfactant according to component c) are SDS, or
said polymer according to component b) are KVA and said surfactant according to component c) are SDS, or
said polymer according to component b) are a mixture of PVP and KVA and said surfactant according to component c) are SDS.

6. The pharmaceutical formulation as claimed in claim 1, characterized in that the pharmaceutical formulation is selected from the group consisting of:
i) a film,
ii) an aerosol,
iii) an aqueous suspension, solution, tincture, cream, paste, lotion, ointment, gel, or capsule releasing these formulations in the oral cavity,
iv) an orodispersible tablet, lozenge or buccal tablet,
the abovementioned formulations preferably being mucoadhesive formulations.

7. The pharmaceutical formulation as claimed in claim 1, wherein the active substance in the formulation can be administered into the bloodstream for a systemic action via the mucosa of the oral cavity or nose.

8. A method for producing a pharmaceutical formulation as claimed in claim 1, wherein comminution of the active substance according to component a) is carried out at least together with the polymer according to component b) and the surfactant according to component c).

9. The method as claimed in claim 8, characterized in that comminution is carried out for a period of 100 to 260 minutes, preferably for a period of 140 to 180 minutes.

10. The method as claimed in claim 8, characterized in that comminution is a milling process, more preferably wet milling, and wherein milling preferably takes place in a stirring ball mill at a peripheral stirrer speed of more than 4 m/s, preferably 5-15 m/s, more preferably 7-11 m/s, particularly preferably 9 m/s.

11. The method as claimed in claim 8, characterized in that further components are added to components a), b), and c) during and/or after the combined comminution thereof.

12. The method as claimed in claim 8, characterized in that
i) after combined comminution of components a), b), and c) in a stirring ball mill, further components to produce a film are added to the stirring ball mill, said further components preferably comprising water-soluble cellulose derivatives,
ii) the resulting total mixture in the stirring ball mill is homogenized, and then
iii) the homogenate obtained is applied to a film as a coating compound or is itself processed into a film.

13. The method as claimed in claim 12, characterized in that the homogenization step ii) is carried out in the stirring ball mill at a peripheral stirrer speed of more than 2 m/s, preferably 3-12 m/s, more preferably 4-8 m/s, particularly preferably 6 m/s.

14. A medicament comprising the pharmaceutical formulation as claimed in claim 1, for use in the treatment of sexual dysfunction, preferably erectile dysfunction.

15. The medicament for use as claimed in claim 14, wherein the maximum serum active substance concentration (tₘₐₓ) is reached within not more than 120 minutes, preferably within not more than 90 minutes, more preferably within not more than 60 minutes, particularly preferably within not more than 45 minutes, after administration of the pharmaceutical formulation.
